(19) United States
(12) Patent Application Publication
(10) Pub. No.: US 2021/0338127 A1
(43) Pub. Date: Nov. 4, 2021

(54) APPARATUS AND METHOD FOR INSERTING ELECTRODE-BASED PROBES INTO BIOLOGICAL TISSUE

(71) Applicant: IMPERIAL COLLEGE OF SCIENCE, TECHNOLOGY AND MEDICINE, London (GB)

(72) Inventors: Matthew L. CAVUTO, London (GB); Timothy CONSTANDINOU, London (GB); Amos G. WINTER, London (GB)

(21) Appl. No.: 17/285,809

(22) PCT Filed: Oct. 30, 2019
(86) PCT No.: PCT/GB2019/053074

§ 371 Date: Apr. 15, 2021

(30) Foreign Application Priority Data
Oct. 31, 2018 (GB) 1817838.4

(51) Int. Cl. A61B 5/25; A61B 34/30

(52) U.S. Cl. CPC A61B 5/25; A61B 2562/028; A61B 34/30

(57) ABSTRACT

A probe assembly comprising: an electrode-based probe comprising a probe head and one or more slender electrodes extending from the probe head for insertion into biological tissue; and a support element disposed around one or more of said electrodes, distal from the probe head, the support element comprising one or more apertures through which said one or more electrodes pass, the probe head and said electrode(s) being movable relative to the support element during insertion; wherein the support element is configured to constrain the angle of the end of the said electrode(s) at the point of insertion into the tissue. Also provided is an insertion device for inserting the electrode(s) of such a probe assembly into biological tissue, the insertion device comprising: means for holding the support element against the tissue or in close proximity to the tissue; and means for applying an insertion force to the probe head, to drive the probe head towards the support element and thereby cause the electrode(s) to move through the aperture(s) in the support element and become inserted into the tissue.

### FIELD OF THE INVENTION

The present invention relates to an apparatus and method for inserting an electrode-based probe into biological tissue. The present invention is particularly applicable, but by no means limited, for use in performing subdural implantation of such a probe into the cortex of the human brain. Furthermore, the present invention is particularly applicable, but by no means limited, for use with electrode-based probes comprising one or more discrete slender electrodes, such as microwires, that are otherwise prone to buckling when an insertion force is applied.

### BACKGROUND TO THE INVENTION

Approximately 1.7% of people in the United States are reportedly living with some form of upper or lower extremity paralysis, according to a 2013 study conducted by the Centers for Disease Control and Prevention. Whether a result of stroke, neurological disorder, or acute traumatic injury, those living with paralysis have had the potential to benefit from the development of brain computer interfaces (BCI) and neuroprostheses since their first demonstrations of restoring movement control in human studies.

However, BCIs have always struggled to achieve chronic recording stability and performance, making them largely infeasible for clinical applications. Most BCI designs operate through the use of penetrating micro-electrode(s), which record functional signals from neurons within the cortex. Issues with chronic recording stability arise from the body's response to damage of brain tissue from micro-electrodes during and after insertion, often referred to as the foreign body response (FBR). The gradual fibrous encapsulation of, and neural cell death around, the BCI ultimately leads to an increase of electrode impedance and loss of usable signal for decoding. Damage caused during insertion, or acute traumatic damage, commonly takes the form of neural cell death and, often more troubling, breeching of the blood-brain barrier (BBB), as cortical vasculature is ruptured. Damage after insertion is largely a result of issues with probe material biocompatibility, electrode-brain modulus mismatch, and the inflammatory response caused by micromotion of the brain tissue against a stiffer and harder electrode. Current gold standards in the field, such as rigid silicone-based arrays, are especially susceptible to tissue encapsulation due to their relatively large size and stiff bulk moduli.

Efforts to minimize both acute and chronic damage from BCI insertion has resulted in the progressive reduction of electrode size and stiffness. Smaller cross-sectional areas of the electrodes allow more flexibility, and softer electrode materials result in less modulus mismatch. Both factors have been shown to not only reduce acute insertional damage, but also chronic inflammation and severity of the FBR. However, a limit is approached in the reduction of both electrode size and modulus, as the electrode must still be able to successfully penetrate the cortex and reach its full insertion depth, without buckling or fracturing. Given the desirable nature of a reliable neural probe, which is flexible enough to largely limit the FBR and achieve chronic recording stability, more and more probes with similarly thin and flexible electrodes are being developed, most running into the same buckling bottleneck. Multiple strategies have been developed to address this limitation, all of which attempt to temporarily stiffen the electrode during insertion, only to then allow the electrode to return to its flexible state for chronic implantation.

These strategies can be largely divided into two main categories: insertion shuttles and bio-dissolvable coatings. The first category uses a stiff support structure or “shuttle”, which is adhered to the flexible electrode during insertion. The shuttle is then separated from the probe and removed from the brain. The second category, bio-dissolvable coatings, involves the use of materials such as sucrose, maltose, gelatin, or polyethylene glycol (PEG). Regardless of material choice, the electrode is first coated in the material, which stiffens it enough to survive insertion, but then dissolves shortly after exposure to the brain tissue. Whether using a shuttle or a dissolvable coating, the main issue is the same. The additional material required to add stiffness to the electrode also increases the cross-sectional area of the electrode during insertion, causing higher degrees of acute tissue damage and ultimately leading to an exacerbated FBR.

Therefore, in order to reduce the level of acute tissue damage and to reduce the FBR, there is a desire to implant electrode-based probes without using insertion shuttles or bio-dissolvable coatings. More particularly, there is a desire to be able to implant electrode-based probes comprising one or more discrete microwires, that are not housed within an insertion shuttle and are not coated in a bio-dissolvable coating. An example of a multi-microwire probe is illustrated in inset (b) of the present FIG. 1. However, the microwires that make up such a probe are inherently prone to buckling when an insertion force is applied.

In relation to this, WO 2017/199052 A2 discloses a neural interface (BCI) system that uses fully wireless probes. FIG. 1(a) of the present application shows the salient features of this system, although the reader is expressly referred to the rest of WO 2017/199052 A2 to fully appreciate it. Preferably the probes have penetrating electrodes that are small and flexible enough to minimize the FBR. Such electrode-based probes may be made up of multiple discrete microwires, for example as shown in inset (b) of the present FIG. 1, with each of the constituent microwires having a diameter of only 20-50 μm, which is small enough to cause minimal tissue damage during insertion and limit the inflammatory response due to micromotion post-insertion. Each constituent microwire is typically around 3-7 mm in length.

However, as with other flexible neural probes in development, such a probe faces difficulties with reliable insertion. For instance, due to their circular cross-section, the constituent microwires of such a probe are prone to buckling in any radial direction. This, in addition to their added flexibility, also makes the electrodes prone to bent and angled insertion, even after successful cortical penetration. While buckling can result in complete failure to penetrate the cortex, bent and angled insertion can result in an equally undesirable outcome, as the electrodes veer off path from their original intended targets. This is referred to as electrode insertion spread, which can lead to neural activity data that is less reliable and more difficult to decode, given the increased distance between neighbouring recording sites.

Thus, in this context, there is a desire to provide a reliable insertion method for an electrode-based probe comprising one or more microwires (preferably multiple microwires), which not only enables successful penetration of the cortex without buckling of the microwire(s), but also achieves, in the case of multi-microwire probes, limited electrode insertion spread. It is further desired that the required insertion forces should be comparable to those of free probe insertion, and that manual insertion (e.g. for the purpose of academic research) should be possible.

### SUMMARY OF THE INVENTION

According to a first aspect of the present invention there is provided a probe assembly comprising: an electrode-based probe comprising a probe head and one or more slender electrodes extending from the probe head for insertion into biological tissue; and a support element disposed around one or more of said electrodes, distal from the probe head, the support element comprising one or more apertures through which said one or more electrodes pass, the probe head and said electrode(s) being movable relative to the support element during insertion; wherein the support element is configured to constrain the angle of the end of the said electrode(s) at the point of insertion into the tissue.

The biological tissue may in particular be that of the human brain, although applications in respect of other types of biological tissue are also possible.

The term “electrode-based probe” as used herein should be interpreted broadly, to encompass both sensing probes (as may be used to detect brain activity, for example) and stimulating probes (as may be used to apply some kind of impulse to the brain, for example).

As principally described herein, the electrodes may be a plurality of discrete microwire electrodes, although the principles of the present work are also applicable to other types and shapes of slender electrodes (which may be singular or plural) that are susceptible to buckling or waywardness during insertion.

By virtue of the support element constraining the end of the said electrode(s), this decreases the susceptibility of the electrode(s) to buckling during insertion, and provides improved control over their path into the tissue. In the case of multi-microwire probes, this also reduces the likelihood of electrode spread, and enables a low insertion force to be used (relative to alternative use of bioresorbable coatings or structure support shuttles). Also, in the case of a singular electrode, this helps ensure that the electrode tip ends up in its target location, rather than veering off path.

In presently-preferred embodiments the support element is configured to constrain the end of the said electrode(s) so as to be orthogonal to the tissue at the point of insertion, i.e. by the apertures in the support element being oriented in such an orthogonal direction. However, for some applications orthogonal insertion may not be desired—for example, when it is desired that the electrodes of a probe should spread evenly and radially from the centre of the probe head during insertion. In such cases, the apertures in the support element may be angled accordingly.

In certain embodiments, to promote linear insertion of the electrode(s) into the tissue, the support element may be configured to constrain the end of the said electrode(s) so as to be linear with the rest of the electrode(s).

In various embodiments the support element may be in the form of a plate, having a thickness sufficient to apply the aforementioned constraint to the end of the electrode(s).

In certain embodiments the shape of the support element corresponds to the shape of the probe head. This lends itself well to the support element being left in place when the probe is fully inserted, with the probe head being on top of the support element (and possibly the probe head and the support element being adhered together after insertion), as the support element occupies the same area on the tissue surface as the probe head. It is also well suited to deployment using an insertion device, as described later.

Preferably the aperture(s) are in the form of one or more discrete holes, through each of which a respective electrode passes. Thus, in the case of a multi-electrode probe, each electrode passes through a respective hole. In the case of a single-electrode probe, the electrode passes through a single hole. In such a manner, the support element provides support and constraint to the/each electrode in all radial directions.

In some embodiments the cross-sectional shape of the aperture(s) may correspond to the cross-sectional shape of their respective electrodes, although in other embodiments this need not be the case.

The shape of the support element may be tailored to accommodate and support electrodes of different lengths. For example, the support element may incorporate a relief cut out to allow shorter electrodes to bypass the plate.

In certain embodiments the underside of the probe head may incorporate one or more protrusions or recesses for engaging with corresponding recesses or protrusions in the upper surface of the support element, thereby enabling the probe head and support element to accurately come into mutual alignment as they come together during the insertion process.

Optionally the support element may be made of a bioresorbable material. Accordingly, such a support element may be resorbed by the body over time, if left in place in the body following the insertion of the probe.

In alternative embodiments, the support element may be a first plate, and the aperture(s) may be in the form of a slot or a plurality of parallel slots within the plate; and the probe assembly further comprises a second such plate, also incorporating a slot or a plurality of parallel slots; wherein the first and second plates are arranged one above the other, such that the slot(s) of the first plate cross the slot(s) of the second plate, the crossing points of the slots defining one or more channels for constraining said one or more electrodes during insertion. By using plates containing such slots, the plates may be removed (by sliding) from around the electrode(s) during the insertion process, so that they need not be left in place when the probe is fully inserted.

In the event that the electrodes are of different lengths, the probe assembly may further comprise one or more additional slotted plates disposed around one or more relatively long electrodes, to provide temporary additional support for the longer electrodes during the insertion process.

To facilitate the removal of such slotted plates from the probe assembly during insertion of the probe, particularly when used with a manual insertion device, each of the slotted plates may comprise a handle for withdrawing the respective plate in a direction parallel to the direction of the slot(s) within the plate. In the case of robotic insertion, the slotted plates may be retracted by servo or some other form of robotic actuation, e.g. in a clinical setting.

According to a second aspect of the invention there is provided a support element for disposal around one or more slender electrodes of an electrode-based probe, the electrode(s) being for insertion into biological tissue, the support element comprising one or more apertures through which said one or more electrodes pass, the electrode(s) being movable relative to the support element during insertion; wherein the support element is usable to constrain the angle of the end of the said electrode(s) at the point of insertion into the tissue.

As mentioned above, in various embodiments the support element may be in the form of a plate, having a thickness sufficient to apply the aforementioned constraint to the end of the electrode(s).

The aperture(s) may be in the form of one or more discrete holes, each for receiving a respective electrode.

Optionally, the support element may be made of a bioresorbable material.

In other embodiments, the aperture(s) may be in the form of a slot or a plurality of parallel slots. The support element may further comprise a handle for withdrawing the plate during the insertion process.

According to a third aspect of the invention there is provided an insertion device for inserting the electrode(s) of a probe assembly in accordance with the first aspect of the invention into biological tissue, the insertion device comprising: means for holding the support element against the tissue or in close proximity to the tissue; and means for applying an insertion force to the probe head, to drive the probe head towards the support element and thereby cause the electrode(s) to move through the aperture(s) in the support element and become inserted into the tissue.

In a presently-preferred embodiment, the insertion device further comprises a device body having a probe-loading tip, the device body having a longitudinal channel therein, in communication with the probe-loading tip; wherein the probe-loading tip is configured to receive and support the probe assembly; wherein the means for holding the support element is provided by the probe-loading tip; and wherein the means for applying an insertion force comprises a plunger located within the longitudinal channel, the plunger having a pushing part at one end, proximal to the probe-loading tip, the plunger being longitudinally advanceable within the channel so as to cause the pushing part to push the probe head in use.

Such an arrangement advantageously provides a controlled linear downward force to the probe head and electrode(s) during the insertion process.

The probe-loading tip may comprise gripping means, such as an O-ring, for gripping the support element during the insertion process.

Further, the probe-loading tip may comprise gripping means, such as an O-ring, for initially gripping the probe head during the insertion process.

To enable controlled separation of the insertion device from the inserted probe at the end of the insertion process, in the presently-preferred embodiment the plunger has a pushable head at the end of the plunger distal from the probe-loading tip, the length of the plunger being such that, when the plunger is fully depressed against the probe head and the support element, the distance by which the underside of the pushable head is proud of the top of the device body is greater than the combined thickness of the probe head and the support element. The device body preferably further comprises a handle (or other lifting means) by which the device body can be raised towards the underside of the pushable head. Accordingly, once the probe has been fully inserted, by holding the pushable head of the plunger against the probe head and simultaneously puffing the device body upwards, towards the underside of the pushable head, the insertion device may be separated from the inserted probe, which is essentially ejected from the end of the probe-loading tip.

With using slotted plates to constrain the electrode(s), as outlined above, the probe-loading tip may comprise lateral slots through which the slotted plates can be inserted to surround the electrode(s) and thereby form the probe assembly, and through which the slotted plates can be withdrawn during insertion of the electrode(s) into the tissue.

In some instances the probe-loading tip may be pre-loaded with the probe assembly, e.g. as a single-use (disposable) insertion device that is ready for use.

Alternatively, the probe-loading tip may be openable to enable successive probe assemblies to be inserted into the probe-loading tip and then deployed into the tissue.

Moreover, the probe-loading tip may be detachable from, and reattachable to, the rest of the device body, thereby enabling successive pre-loaded probe-loading tips to be used with a single device body.

The insertion device may be for manual use, as primarily described herein, although it may readily be adapted for robotic actuation, as those skilled in the art will appreciate.

According to a fourth aspect of the invention there is provided a probe-loading tip pre-loaded with a probe assembly in accordance with the first aspect of the invention, for use in an insertion device in accordance with the third aspect of the invention.

According to a fifth aspect of the invention there is provided a method of inserting one or more electrodes of an electrode-based probe into biological tissue, using a probe assembly in accordance with the first aspect of the invention and/or an insertion device in accordance with the third aspect of the invention.

### BRIEF DESCRIPTION OF THE DRAWINGS

Embodiments of the invention will now be described, by way of example only, and with reference to the drawings in which:

FIG. 1 shows (a) a cross-sectional schematic diagram of the system of WO 2017/199052 A2 (taken from FIG. 1 of WO 2017/199052 A2), and (b) an enlarged illustration of a multi-microwire probe, suitable for use in the system of WO 2017/199052 A2 and as considered in the present work, with approximate exemplary dimensions ascribed;

FIG. 2 is an isometric view of a probe insertion device according to an embodiment of the invention, having a syringe-like (manually-operated) form with a two-part probe-loading tip;

FIG. 3 is a partially-exploded view of the device of FIG. 2, together with an anti-buckling (hereafter “AB”) multi-microwire probe according to an embodiment of the invention having an AB plate to constrain the microwires;

FIG. 4 is a cross-sectional illustration of a stepwise probe insertion procedure employing the device of FIGS. 2 and 3, the dashed line representing the surface of the biological tissue (e.g. grey matter of the brain) into which the AB probe is inserted;

FIG. 5 shows the stepwise probe insertion procedure in respect of the AB probe architecture alone, the dashed line again representing the surface of the biological tissue into which the AB probe is inserted;

FIG. 6 illustrates how, in relation to the formula for Euler's Critical Load (Equation 1 below), the column effective length factor, K, changes with varying various column end conditions, namely (a) rotation fixed and translation free (top), rotation free and translation fixed (bottom), (b) rotation fixed and translation free (top), rotation fixed and translation fixed (bottom), and (c) rotation fixed and translation fixed (top), rotation fixed and translation fixed (bottom);

FIG. 7 shows photographs of the three probe types that were manufactured and tested, namely (a) a free probe, with unsupported electrodes, (b) an AB probe with an AB plate, and (c) a sucrose-coated probe, with electrodes coated in sucrose via drawing lithography;

FIG. 8 presents sample photographs of (a) an inserted free probe, and (b) an inserted AB probe;

FIG. 9 is a scatter plot showing insertion force and electrode tip spread for the three different probe types shown in FIG. 7 (ten of each) into 0.6% agarose gel, with mean values for each probe type being shown by the enlarged respective symbol;

FIG. 10 shows mean peak insertion force for the three different probe types shown in FIG. 7;

FIG. 11 shows mean electrode tip spread for the three different probe types shown in FIG. 7;

FIG. 12 shows mean insertion depth for the three different probe types shown in FIG. 7;

FIG. 13 is an isometric view of an alternative probe insertion device which uses multiple slidable slotted AB plates to constrain the microwires of a multi-microwire probe;

FIG. 14 is an isometric cross-sectional view of the device of FIG. 13, showing a probe mid-insertion, with one of the slotted AB plates removed (prematurely in this case, but done so for illustrative purposes);

FIG. 15 is an isolated view of the probe and slotted AB plates of FIGS. 13 and 14;

FIGS. 16a and 16b illustrate an alternative arrangement in which multiple slotted AB plates are used to constrain different lengths of electrodes, the AB plates being sequentially removed (by being slid transversely) as the probe is inserted;

FIG. 17 illustrates, in (a) an isometric view, and (b) a plan view from below, an alternative arrangement in which a single AB plate is used to constrain different lengths of electrodes, with a relief cut out to allow shorter (and less prone to buckling) electrodes to bypass the AB plate;

FIG. 18 illustrates an alternative arrangement of a multi-thickness probe head and a counterpart multi-thickness AB plate, which fit together in an interlocking manner (like a puzzle) when fully inserted, the multiple steps of the probe head and the AB plate being used to accommodate and constrain different lengths of electrodes; and

FIG. 19 shows the stepwise insertion procedure of the probe and AB plate of FIG. 18.

In the figures, like elements are indicated by like reference numerals throughout.

### DETAILED DESCRIPTION OF PREFERRED EMBODIMENTS

The present embodiments represent the best ways known to the Applicant of putting the invention into practice. However, they are not the only ways in which this can be achieved.

### INTRODUCTION

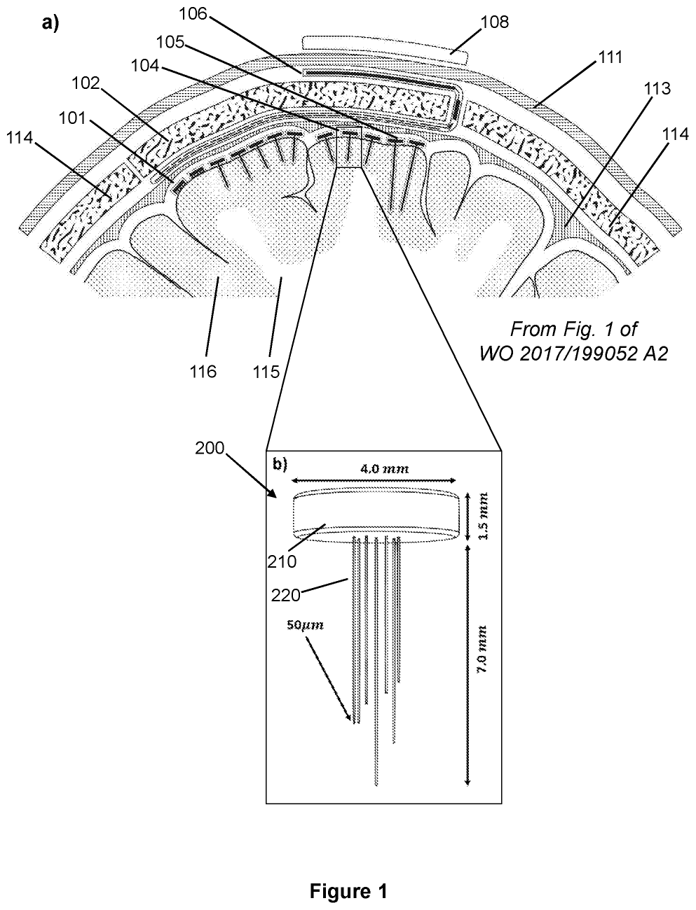

By way of introduction, FIG. 1(a) is a cross-sectional schematic diagram of the system of WO 2017/199052 A2 (taken from FIG. 1 of WO 2017/199052 A2), with which the present work is applicable. It should be noted, though, that the present work is in no way limited to use with the system of WO 2017/199052 A2, and may be applied to any electrode-based probe comprising one or more discrete slender electrodes that are otherwise prone to buckling when an insertion force is applied.

For anatomical context, the human head has an outer surface of skin/tissue/scalp layers 111, beneath which is the skull 114. Beneath the skull 114 is the dura mater (or simply “dura”) 113. Under the dura 113 is the brain, which is made up of white matter 115 and grey matter 116.

A section 102 of the skull is removed by a surgeon for installation of the system of WO 2017/199052 A2, and then returned to position afterwards.

The system of WO 2017/199052 A2 includes a plurality of implantable wireless probes 101, 104, 105, which, in use, are surgically implanted into the brain, beneath the dura 113. Probe 101 is for surface monitoring micro-electrocorticography (micro-ECoG), and is positioned on the surface of the grey matter 116. Probes 104, 105 are for intracortical recording by penetrating into the grey matter 116. Probe 104 has a relatively short shank length. Probe 105 has a longer shank length, to reach deeper into the grey matter 116.

Above the skin/scalp 111, an external transceiver device 108 is provided to transmit power and control signals to the implanted wireless probes 101, 104, 105, by means of a transponder device 106. More particularly, the transponder device 106 comprises a primary coil (above the skull) for receiving power and control signals from the external transceiver device 108, the primary coil being connected to an array of smaller coils (beneath the skull, above the dura) for transmitting power and control signals to the wireless probes 101, 104, 105.

Other features of the present FIG. 1 are explained in greater detail in WO 2017/199052 A2, through reference to FIG. 1 of that document.

The multi-microwire probes of the present work may be used as the wireless probes 104, 105 of WO 2017/199052 A2.

FIG. 1(b) is an enlarged illustration of a multi-microwire probe 200, suitable for (but not limited to) use in the system of WO 2017/199052 A2, with approximate exemplary dimensions ascribed. The probe 200 comprises a head 210 and a plurality of microwire electrodes 220, although the principles of the present work also apply to a probe having a single microwire electrode 220. Further details in respect of the construction and functionality of the head of such a probe, and the operation of such a probe more generally, may be found in WO 2017/199052 A2.

Overview

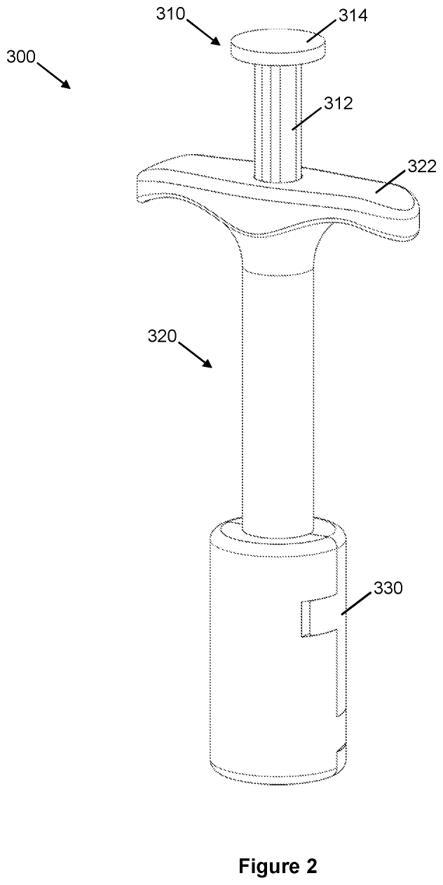

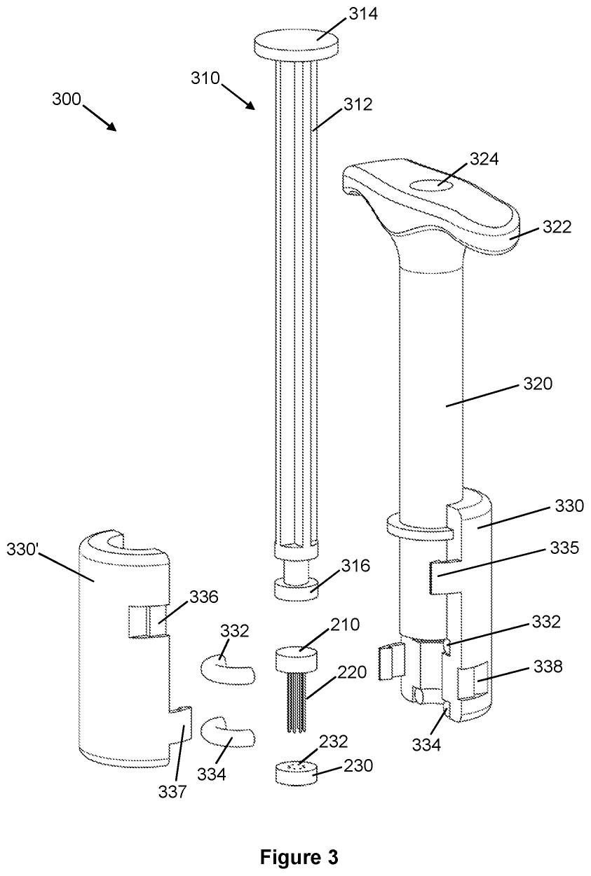

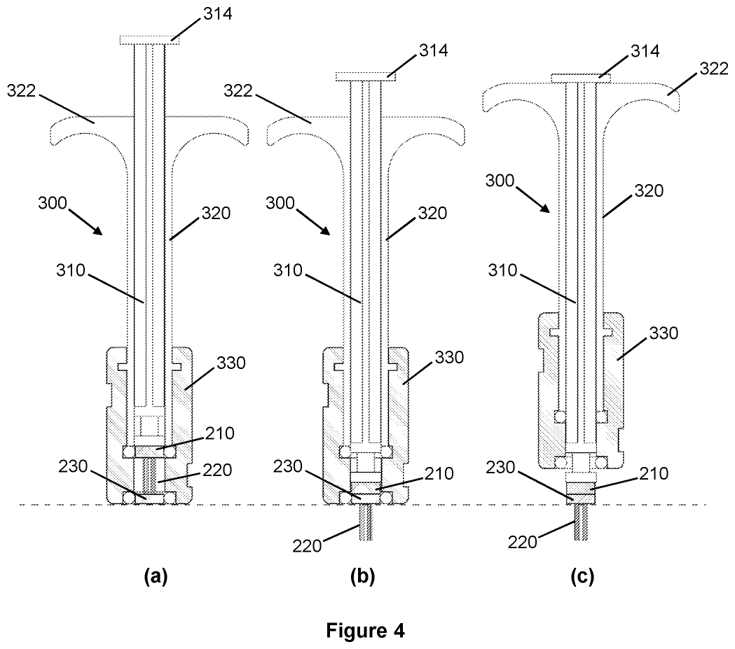

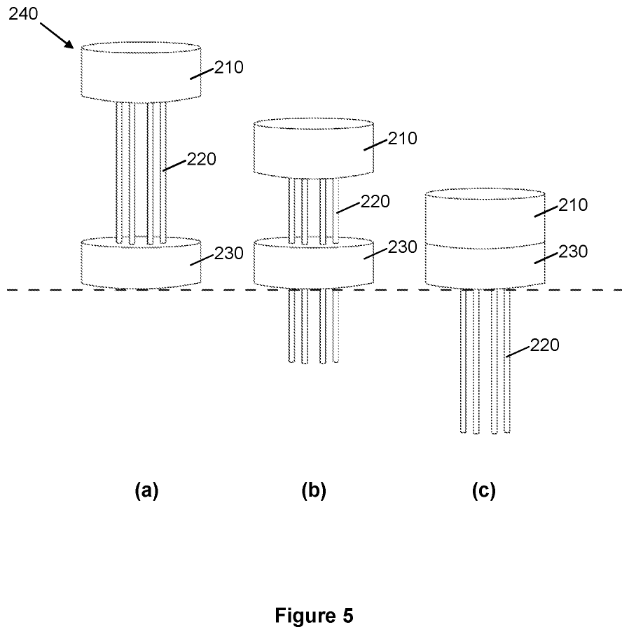

As described in greater detail below, the present work provides an insertion device (e.g. as shown in FIG. 2) and a modular probe structure (e.g. as shown in FIGS. 3, 4 and 5), together with a method of inserting such a probe into biological tissue such as the brain (e.g. as shown in FIGS. 4 and 5). The method is shown to achieve significantly less electrode insertion spread, when compared to insertion without the insertion device and the new probe architecture, while maintaining a low insertion force relative to alternative use of a bio-dissolvable sucrose coating.

AB Plates

To prevent electrode insertion spread and buckling of the microwire electrodes 220 of the probe 200, while avoiding an increase in the insertion force, use of a shuttle or bio-dissolvable coating was ruled out due to their inevitable addition of inserted cross-sectional area. Instead, the present work introduces anti-buckling insertion guides, hereafter referred to as anti-buckling (“AB”) plates, to prevent electrode insertion spread and buckling.

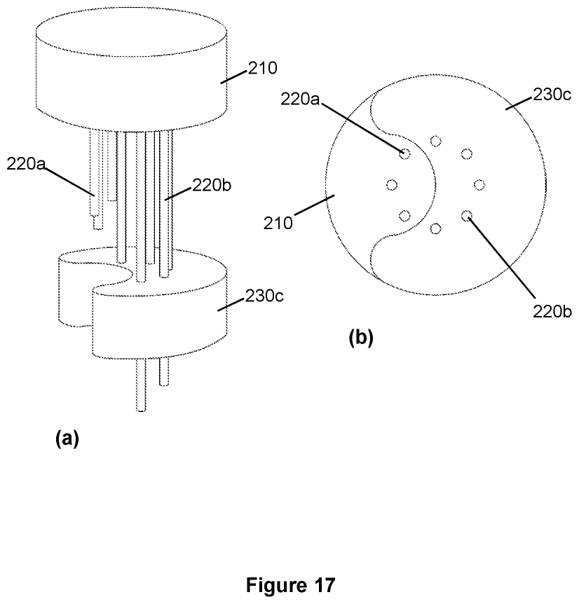

An AB plate is a support element disposed around the electrodes 220 (or a subset of the electrodes, e.g. as described below with reference to FIG. 17), distal from the probe head 210, and comprising one or more apertures through which the electrodes pass. The probe head 210 and the electrodes 220 are movable relative to the AB plate during insertion of the electrodes into the tissue. The AB plate is configured to constrain the ends of the electrodes so as to be at a desired angle (in the presently-preferred embodiments, orthogonal) to the tissue at the point of insertion.

For the purpose of testing and development, a cylindrical neural probe head 210 was used, having a diameter of 4.0 mm and a thickness of 1.0-2.5 mm, with eight niobium microwire electrodes 220 protruding from the centre, each with a diameter of 50 μm and a length of 7.0 mm. Thus, the probe head 210 has a circular cross-sectional geometry. The microwire electrodes 220 are substantially straight and substantially parallel to each other, and extend substantially perpendicular to the probe head 210.

The cross-sectional (plan view) shape of the AB plate corresponds to that of the probe head 210, and is therefore circular in the presently-described embodiments. However, as those skilled in the art will appreciate, probe heads of other cross-sectional geometries (e.g. square, rectangular, triangular, hexagonal, etc.) are also possible. In such cases, AB plates of corresponding geometries can readily be designed.

Given the circular cross-section of each of the microwire electrodes 220, buckling and bending in any radial direction can potentially occur, and thus the AB plate(s) are designed to provide load support around the entire circumference of each microwire electrode 220.

In one embodiment, as shown in FIGS. 3, 4 and 5, this can be accomplished using a single AB plate 230, having the same outer geometry (in this case, circular) as the probe head 210, and incorporating holes 232 for each microwire electrode 220 to pass through. As illustrated, the AB plate 230 has a flat upper surface and a flat underside, parallel to the upper surface.

In this embodiment, to provide optimum support for each microwire electrode 220 in all radial directions, each microwire electrode 220 passes through a discrete respective hole 232 in the AB plate 230, with the diameter of each hole being only slightly larger than the respective electrode. Accordingly, this provides constraint to the electrode 220 around the whole of its circumference, whilst allowing the electrode to move freely through the AB plate 230 during the insertion process. In the illustrated embodiment the holes 232 are orthogonal to the underside (and upper surface) of the AB plate 230, to thereby constrain the ends of the electrodes 220 so as to be orthogonal to the tissue at the point of insertion. However, in alternative embodiments the holes 232 may be angled differently, e.g. to cause the electrodes 220 to spread from the centre of the probe head 210 during insertion.

In other variants, more than one microwire electrode 220 may pass through a common hole or slot in the AB plate.

Prior to insertion, the AB plate 230 is positioned around the microwire electrodes 220, such that the bottoms of the electrodes 220 are contained within the holes 232 of the AB plate 230. Whilst the electrodes 220 may all be of the same length, as illustrated, this need not be the case. In the event that the electrodes 220 are of different lengths, the bottoms of at least some of the electrodes 220 are contained within the AB plate 230.

Preferably (as shown in FIG. 5(a)) the probe 200 is supplied with the AB plate 230 already in place around the bottoms of the electrodes 220, i.e. as a probe assembly 240. Such a probe assembly may be referred to as an AB probe.

For insertion of the electrodes 220 of the probe 200 into tissue, the bottom surface of the AB plate 230 (containing the bottoms of the electrodes 220) is brought close to, or in contact with, the target area of the tissue (e.g. cortex). The probe head 210 is then driven downward (FIG. 5(b)), with the microwire electrodes 220 sliding through the holes in the AB plate 230 and into the tissue. At the point of full insertion (FIG. 5(c)), the probe head 210 comes into contact with the AB plate 230. During insertion, the AB plate 230 acts to ensure orthogonality of the electrodes 220 to the surface of the tissue, as well as bearing the load of any electrode bending/buckling deflection across its thickness.

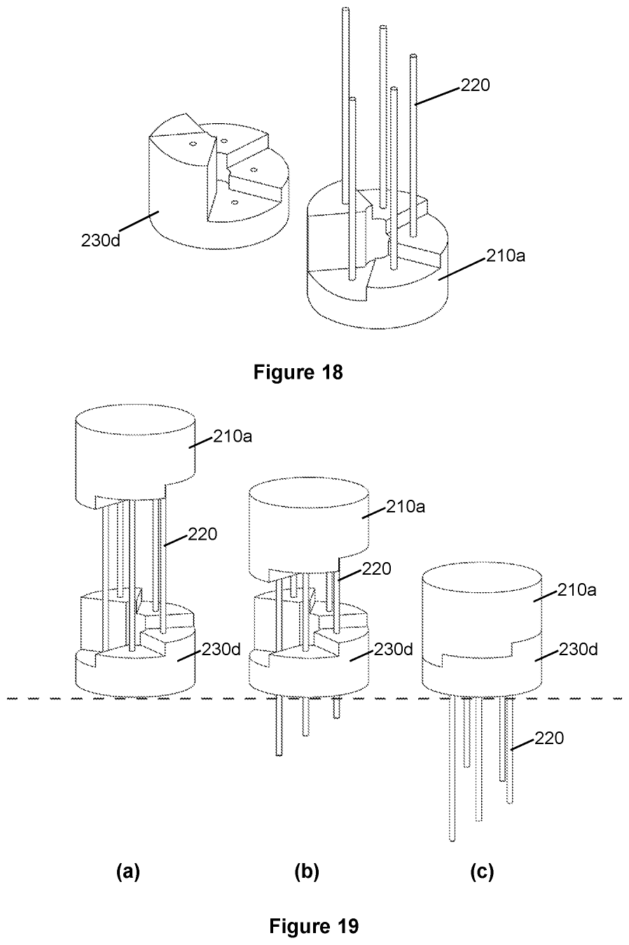

In the present embodiment the underside of the probe head 210 (i.e. the side which ultimately contacts the AB plate 230) is flat. Similarly, the upper face of the AB plate 230, which ultimately contacts the underside of the probe head 210, is also flat. However, in alternative embodiments, the underside of the probe head 210 may be profiled with one or more protrusions or recesses, for engaging with corresponding recesses or protrusions in the upper surface of the AB plate 230 as the probe head 210 and the AB plate 230 come together. A version of such an arrangement is introduced below with reference to FIGS. 18 and 19.

The lower face of the AB plate 230, which contacts the tissue, is preferably also flat, although it may alternatively have some other surface profiling.

### Insertion Device

To achieve controlled insertion of the electrodes 220 into the tissue using the AB plate 230, as shown in FIGS. 2, 3 and 4 the present work provides a syringe-like insertion device 300 for probe insertion. The insertion device 300 comprises a plunger 310, and a hollow body 320 having a probe-loading tip 330. The plunger 310 comprises a longitudinal shaft 312 having a pushable head 314 (which may be pushed by a user, e.g. applying finger pressure) at one end, and a probe-pushing part 316 at the other end. The body 320 includes a handle 322 by which the insertion device can be supported by a user, and a longitudinal channel 324 in which the shaft 312 of the plunger 310 locates. The plunger 310 is movable relative to the body 320, by virtue of the shaft 312 of the plunger 310 being longitudinally advanceable within the channel 324.

In use, the probe-pushing part 316 of the plunger 310 contacts and pushes the probe head 210, to insert the probe 200 (specifically, the electrodes 220 thereof) into the tissue. To apply even pressure to the probe 200, the geometry of the probe-pushing part 316 corresponds to that of the probe head 210. Thus, in this embodiment, the probe-pushing part 316 has a circular cross-sectional shape, to match the circular shape of the probe head 210, but other geometries are possible (e.g. square, rectangular, triangular, hexagonal, etc.) as outlined above.

In the present embodiment the underside of the probe-pushing part 316 (i.e. the side which contacts the probe head 210 to push it) is flat. However, the underside of the probe-pushing part 316 may alternatively be profiled with one or more protrusions or recesses, to engage with corresponding protrusions or recesses in the probe head 210.

In the illustrated embodiment the plunger shaft 312 has a cross-shaped cross-section along much of its length, to reduce friction with the walls of the channel 324, but in alternative embodiments the plunger shaft 312 may have other cross-sectional geometries.

In the illustrated embodiment the longitudinal channel 324 has a circular cross-section, shaped to allow the plunger shaft 312 to pass along it, whilst supporting and laterally constraining the plunger shaft 312. Thus, the cross-sectional geometry of the longitudinal channel 324 closely corresponds to the maximum external cross-sectional geometry of the plunger shaft 312.

The probe-loading tip 330 is shaped to receive and support a probe 200 with an AB plate 230 already in place around the bottoms of the electrodes 220 (i.e. an AB probe 240).

The probe-loading tip 330 provides a sliding bearing surface for the outer circumferences of both the probe head 210 and the AB plate 230. Preferably, as illustrated, the probe-loading tip 330 includes a first O-ring 332, for initially gently gripping the probe head 210, and a second O-ring 334, for gently gripping the AB plate 230.

As shown most clearly in FIG. 3, a detachable part 330′ of the probe-loading tip 330 may be removed to enable the user to introduce the AB probe 240 with the AB plate 230 in place around the bottoms of the electrodes 220. The detachable part 330′ may then be refitted (e.g. by engaging clips 335 and 337 with their respective recesses 336 and 338; similar clips are provided on the reverse side of the probe-loading tip 330) once the AB probe 240 is in place.

As illustrated, each of the O-rings 332, 334 may be provided in two halves which come together when the detachable part 330′ of the probe-loading tip 330 is fitted into place.

Such an insertion device 300 can be used a number of times, to insert multiple probes. Alternatively, a single-use insertion device 300 may be supplied with an AB probe 240 pre-loaded within the probe-loading tip 330.

Moreover, the probe-loading tip 330 may be detachable from, and reattachable to, the rest of the device body 320, thereby enabling successive pre-loaded probe-loading tips to be used with a single device body. Accordingly, a user may obtain multiple pre-loaded probe-loading tips for use with a single device body.

Probe insertion is accomplished by means of the plunger 310 being advanced down the channel 324 within the body 320, under the application of pressure to the pushable head 314, such that the probe-pushing part 316 contacts the top of the probe head 210 and applies a linear downward force to it.

FIGS. 4 and 5 illustrate the probe insertion procedure in more detail, with the dashed line representing the surface of the biological tissue (e.g. grey matter of the brain) into which the probe is inserted. FIG. 4(a) shows the first step of the insertion process, during which the user (e.g. a surgeon) gently brings a loaded insertion device 300 into contact with (or just above) the tissue surface, the user holding the insertion device 300 in that position using the handle 322. This is so as to position the AB plate 230 in contact with the tissue surface (or just above the tissue surface; the AB plate 230 might only come into contact with the tissue surface at the end of the insertion procedure—although if it is slotted, as described below, it may be removed during the insertion procedure). FIG. 5(a) is a close-up of the AB probe 240 in isolation, at the same stage of the process.

Next, the user depresses the plunger 310 by applying pressure on the pushable head 314, driving the probe head 210 downward towards the AB plate 230, and thereby implanting the microwire electrodes 220 into the tissue. Partial insertion of the microwire electrodes 220 is depicted in FIG. 5(b), with there being a gap between the underside of the probe head 210 and the upper surface of the AB plate 230. Full insertion of the microwire electrodes 220 is depicted in FIG. 4(b) and FIG. 5(c), with the underside of the probe head 210 having come into contact with the upper surface of the AB plate 230.

Lastly, as shown in FIG. 4(c), the embedded probe is separated from the probe-loading tip 330 by the user gently holding the pushable head 314 of plunger 310 in the depressed position against the probe head 210 and simultaneously pulling the body 320 upwards, by means of the handle 322. Accordingly, the body 320 (and with it the probe-loading tip 330) moves away from the embedded probe, towards the underside of the pushable head 314, thereby releasing the AB plate 230 (and the probe head 210) from the second O-ring 334, and enabling the insertion device 300 to be removed from the insertion site.

It will be appreciated that, when the plunger 310 is fully depressed against the probe head 210 and the AB plate 230, as in FIG. 4(b), the distance by which the underside of the pushable head 314 is proud of the top of the body 320 is slightly greater than the combined thickness of the probe head 210 and the AB plate 230. As a consequence, subsequently raising the body 320 so as to come into contact with the underside of the pushable head 314 is sufficient to fully separate the probe-loading tip 330 from the embedded probe, as shown in FIG. 4(c).

### Theoretical Analysis

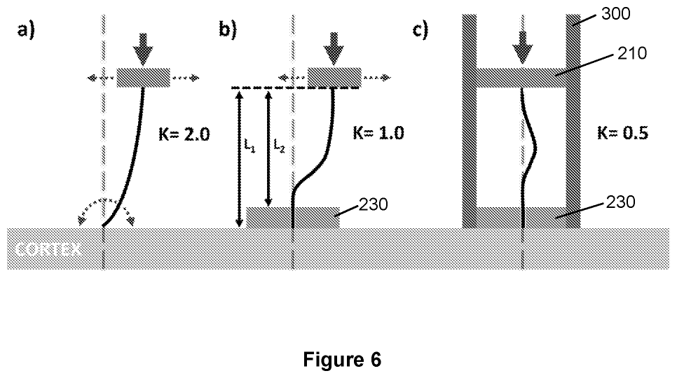

With reference to FIG. 6, the formula for Euler's Critical Load (Equation 1) can be used to determine the maximum load F_{b} that a column in compression can withstand before buckling. This formula can be applied to the buckling of a microwire, the microwire being considered to be a slender column. For the microwire of a probe not to buckle on application of an insertion force, F_{b} must be greater than the insertion force that causes the microwire to be pushed into the tissue below.

The maximum load F_{b} is given by

$${F}_{b} = \frac{{π}^{2} IE}{{( KL )}^{2}} [ N ] \qquad ( 1 )$$

where I is the moment area of inertia of the column cross-section, E is the Young's modulus of the material, K is the column effective length factor, and L is the unsupported length of the column.

Thus, to increase the critical load F_{b}, aside from increasing E (which is a material property and thus taken to be constant), one can also decrease K or L, the column effective length factor and the unsupported length of the column respectively. (While increasing I, the moment area of inertia of the column cross-section, would also increase the critical load, changing the electrode cross-section is difficult on the relevant scale, and so I is also taken to be constant for a given electrode type.)

FIG. 6 illustrates how K changes with varying column end conditions, namely (a) rotation fixed and translation free (top), rotation free and translation fixed (bottom); (b) rotation fixed and translation free (top), rotation fixed and translation fixed (bottom); and (c) rotation fixed and translation fixed (top), rotation fixed and translation fixed (bottom). The thicker solid arrows represent the applied force direction, while the dashed arrows represent extraneous degrees of freedom contributing to K. L₁ and L₂ represent the unsupported column length without and with the anti-buckling insertion guide respectively.

The present work achieves a decrease in both K and L. First, K is decreased by constraining the tip of the electrode orthogonally to the surface of the cortex with the addition of an AB plate, which acts as a stencilled insertion guide for the electrode. Furthermore, the new effective length, L₂, of the electrode under load bearing is decreased from the original length, L₁, by the thickness of the plate (FIG. 6(b)).

More particularly, the use of an AB plate 230 alters the bottom end constraint of each microwire 220 from that of FIG. 6(a), i.e. “rotation free and translation fixed”, to that of FIG. 6(b), i.e. “rotation fixed and translation fixed”, thereby reducing the column effective length factor K from roughly 2.0 to 1.0, and theoretically doubling the critical buckling load.

Furthermore, the insertion device 300 enables K to be further reduced by also constraining the relative motion of the probe head 210 and AB plate 230 to linear motion in the z-axis, as per FIG. 6(c), thus introducing a top end constraint of “rotation fixed and translation fixed”. This further decreases the theoretical K value from 1.0 to 0.50, resulting in a 4-fold increase in critical buckling load over free manual insertion.

### Slotted AB Plates

It should be noted that, with the AB probe illustrated in FIGS. 3 to 5, the AB plate 230 is not removed prior to full insertion, and instead bonds to the probe head 210 through an adhesive layer on the top surface of the AB plate 230. This was required due mainly to the possibility of the cylindrical microwires 220 buckling in any direction, necessitating closed holes in the AB plate 230, instead of open slots. As a result, this design does not affect the unsupported column length (L), as the unsupported length of electrode between the AB plate 230 and the probe head 210 must still match the entire electrode length of equivalent “free probe” electrodes, to reach the same final insertion depth. Alternatively, one could take advantage of decreasing L by, instead of using one plate, using two slotted AB plates, wherein the parallel slots of one plate cross the parallel slots of the other plate, preferably orthogonally, such that each plate constrains a single axis of buckling on its own. Such a slotted design allows for the removal of the AB plates prior to complete insertion, avoiding the need to make the electrodes longer than their required insertion depth. AB plates employing such a slotted design are described in greater detail below, together with a corresponding insertion device.

A probe with a single AB plate, slotted AB plates, or some other arrangement, may be applied to robotic insertion. Robotic insertion may involve pneumatic or servo-controlled actuation of the plunger, providing a precise and consistent insertion speed. The robotic apparatus may well not resemble the manual device shown in FIGS. 2 and 3, although it may employ the same principles. A manual device, consisting of a familiar syringe form factor (e.g. as shown in FIGS. 2 and 3), would be convenient on the other hand for academic insertion studies, in which an expensive robotic system cannot be justified. In both cases, the design is easily adaptable for use within a stereotactic frame, allowing for precise location.

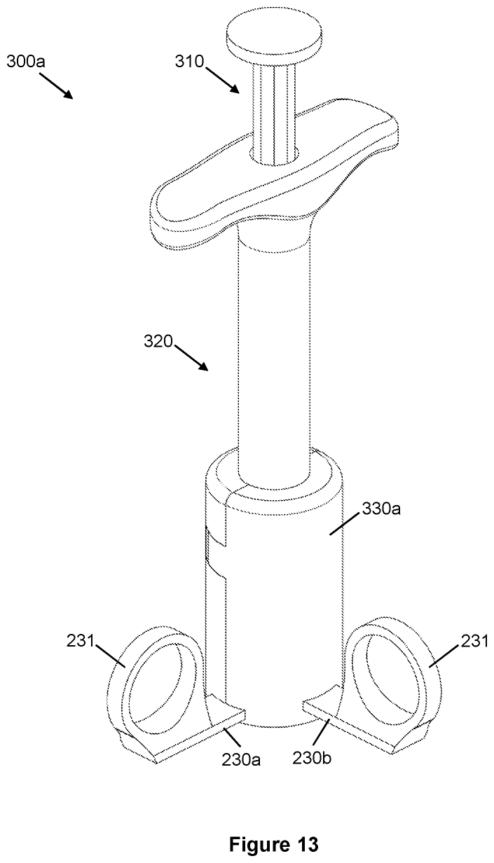

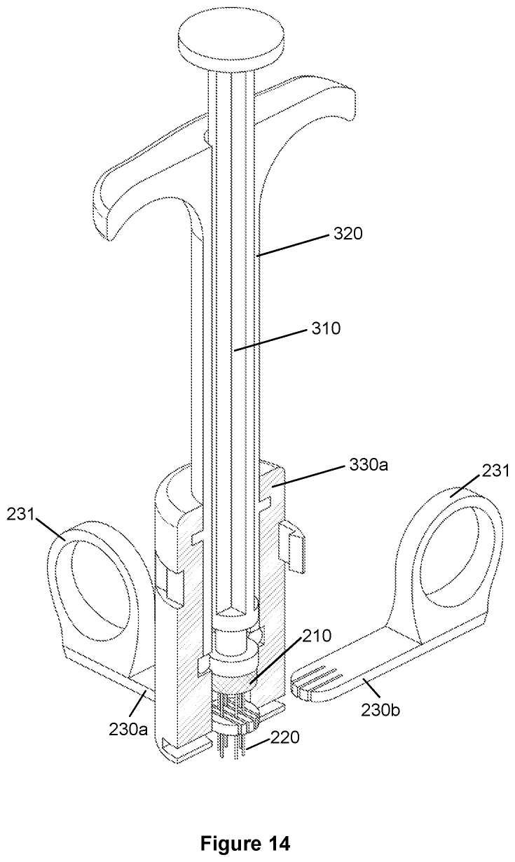

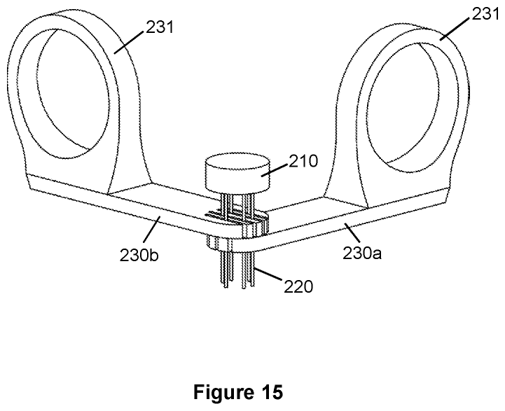

Indeed, all the described embodiments, whether in the context of a singular perforated plate, multiple slotted plates, or some other arrangement, are readily adaptable for automated or semi-automated robotic insertion. In this scenario, precise positioning of the insertion device and actuation of the plunger could be servo controlled, allowing for a more efficient and accurate procedure. With respect to embodiments using slotted plates (e.g. as illustrated in FIGS. 13-16, as described below), the timed and sequential removal of two or more slotted plates could also be automated through robotic control. Note further that the particular physical implementation of the device for both manual and robotic insertion could be different. For example, disposable cartridges, each pre-loaded with an AB probe assembly, could simply be inserted into a robotic positioning frame, comprising a permanent plunger actuation module. Following each insertion, a new cartridge could be loaded, cutting down on the cost of utilizing a disposable plunger and barrel assembly. Other implementations can also be envisaged, as those skilled in the art of surgical robotics or neurosurgery will appreciate.

Device Prototype Construction

A prototype insertion device (of the form illustrated in FIG. 2) was constructed using a 1.0 mL disposable veterinary syringe as the main device body 320. The tapered tip of the veterinary syringe was pared off to leave a hollow cylinder of constant diameter and the freshly cut tip was finished smoothly with a heat gun. To allow for easy loading of the AB probes, a two-part tip probe-loading tip 330 was 3D-printed with a Markforged Mark Two printer, using its proprietary nylon-based Onyx filament. As shown in FIG. 3, within each half of the probe-loading tip 330 are two recesses, each holding half of a respective O-ring 332, 334. The O-rings 332, 334 were fixed into place with cyanoacrylate adhesive. When assembled with a loaded AB probe 240, the two halves of the top O-ring 332 gently grip the probe head 210, while the two halves of the bottom O-ring 334, flush with the bottom face of the probe-loading tip 330, grip the AB plate 230. This ensures that the probe head 210 and AB plate 230 are fixed at the correct distance from each other prior to insertion. When loaded, the two halves of the device tip are held together by flexible clips (e.g. 335 and 337), to allow for convenient assembly and disassembly. While, for the benefit of cost, the probe-loading tip 330 of this prototype has a two-part reloadable design, the device could instead be disposable. In this case, after each insertion, the empty device would be discarded, and a new preloaded device used for the next insertion. This would be particularly convenient for clinical applications, not only decreasing procedure time, by not having to reload a probe for each insertion, but also maintaining a higher degree of sterility.

Mack Probe Construction

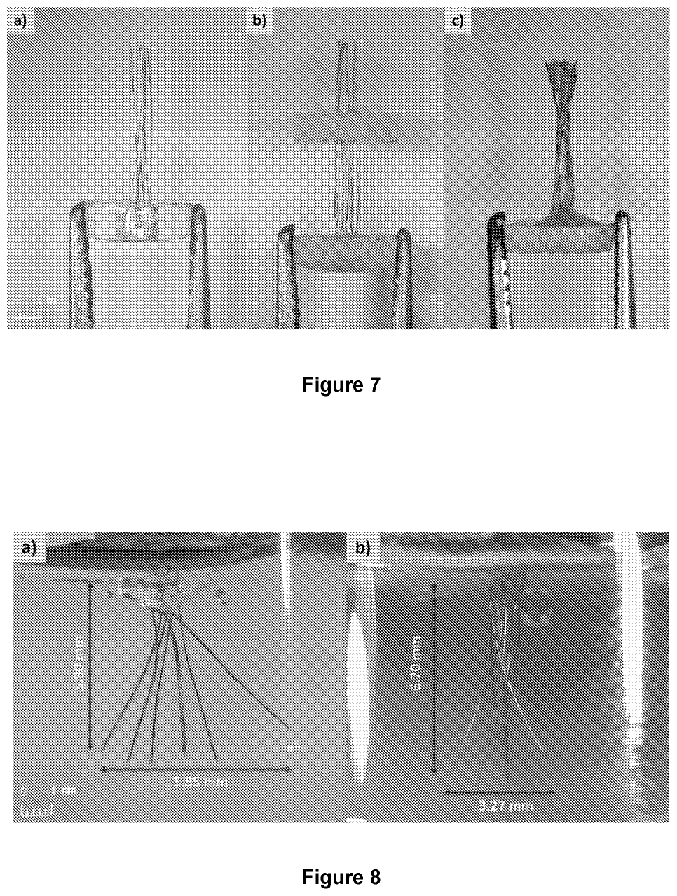

To validate the device's performance, three types of mock probes were constructed, as shown in FIG. 7, namely (a) free probes, with unsupported electrodes; (b) AB probes with an AB plate, and (c) sucrose-coated probes, with electrodes coated in sucrose via drawing lithography. In each case, the mock probe consists of a cylindrical extruded acrylic head with eight 50 μm diameter niobium microwire electrodes protruding from the centre. The electrodes were patterned with a single electrode in the centre of the probe head and seven electrodes evenly spaced in a 1.0 mm diameter concentric circle. Further details for each specific probe type are as follows:

Free probe construction: Probe heads were cut from a sheet of 1.0 mm thick extruded acrylic with a 10W CO₂ laser (VSL2.30, Universal Laser Systems, Scottsdale, Ariz., USA). The power setting was set to −45% under the extruded acrylic material profile to achieve the thinnest curf and smallest diameter holes possible. At this setting, the resultant holes were tapered from approximately a diameter of 150-175 μm at the top face to 75-100 μm at the bottom. Each probe head was then placed on a depth gauge jig, consisting of a 1.5 mm diameter hole that was 7.0 mm deep, to control both the lengths and alignment of the protruding microwires. Tesa® double stick tape was used to gently hold the probe body centred over the jig, while microwires were placed in each of the eight tapered holes. With the microwires in place, a drop of Loctite 406 low viscosity cyanoacrylate was placed on the top surface of the disk and allowed to wick into the tapered holes through capillary action. Loctite SF 7457 cyanoacrylate activator was then applied to quickly cure the adhesive and fix the wires in the probe head. Excess wire was then clipped and sanded flush.

Sucrose-coated probe construction: Coating free probes in sucrose was accomplished through a process referred to as drawing lithography. 10 g of sucrose was dissolved in 10 mL of distilled water with the aid of a magnetic stirrer and hot plate. The mixture was heated to approximately 100° C. for 20 minutes, allowing an appropriate amount of water to evaporate before being removed from the hot plate to cool. Using a stand and pair of forceps, the probe was the dipped into the cooling solution up to the bottom surface of the probe head. Once the temperature reached approximately 75° C., the glass transition temperature of sucrose, the probe was slowly pulled from the solution, resulting in an even hardened coating of sucrose left surrounding the eight microwire electrodes. Excess sucrose was trimmed from the tip and the probes were left to fully solidify in a freezer for one hour. The temperature was continuously monitored throughout the entire process with Sentron S1400 combination pH/temperature probe.

AB probe construction: AB probes were made through a similar process to free probes, but with a few key differences. First, a bilayer stack of 1.0 mm acrylic on top of 1.5 mm acrylic, held together with Tesa® double stick tape, was used to cut both the AB plates and probe heads at the same time, ensuring precise alignment of the microwires through the holes. Each bilayer cylinder was then placed on top of the depth gauge jig, with the 1.5 mm layer on the bottom. Microwires were then placed in each hole as before, however not immediately fixed in place with cyanoacrylate adhesive. First, with the microwires in place, the two halves of acrylic were carefully separated, and two 22-gauge wires were slid in between the two halves. It was important to maintain this gap in the two halves during adhesive application to prevent the cyanoacrylate from wicking through both layers of acrylic and fixing the AB plate to the microwires. Post curing, the extra protruding wire was finished flush to the probe head as before.

Wire straightening: Note that prior to cutting segments of microwire from the spool for placement in the probe bodies, 20 cm segments were straightened with the aid of a microwire straightening jig. Straightening was accomplished through applying a fixed tension on the wire, 200 grams of force (i.e. 1.96 N) for 50 μm niobium.

Experimental Setup and Procedure

Ten of each of the above three probe types were evaluated by recording maximum insertion force, electrode tip spread, and average insertion depth in 0.6% by weight agarose gel, which was used to simulate cortical tissue. For this, agarose powder was dissolved in distilled water with a hot plate and magnetic stirrer at a temperature of 100° C. for 20 minutes and poured into 15 mm diameter wells, which were then allowed to cool for 1 hour at room temperature. Fixtures were 3D printed to interface with a single column micromanipulator (5543, Instron, Norwood, Mass., USA), allowing for controlled insertion rate. The fixtures were used to mount a precision gram load cell (S256-10 g, Strain Measurement Devices, Chedburgh, England), on which an agarose well was placed for each test. For all tests, the probes were inserted at a rate of 600 μm/s to a depth of 4.5 mm into the agarose gel phantom, while the force was recorded at a sample rate of 100 Hz using a USB strain converter and accompanying logging software (DSCUSB, Applied Measurements, Berkshire, England).

Two fixtures were used; one for testing the free and sucrose-coated probes, and one for testing the AB probes in conjunction with the insertion device 300. This was necessary due to the added complexity of gripping and actuating the insertion device 300.

The fixture for testing the free and sucrose-coated probes was mounted on the top bracket of the Instron machine, which moved down toward an inverted probe resting on a fixed plate. This orientation avoided the need to use double stick tape to suspend each probe from the top bracket, allowing easier removal of the agarose gel well with the embedded probe for later imaging and measurement of electrode tip spread and insertion depth.

The fixture for the AB probe and insertion device testing was mounted in the bottom bracket of the Instron machine. A square frame was used to both mount the load cell and grasp the insertion device such that the tip of the electrodes (and bottom of the AB plate) were suspended just above the surface of the agarose gel. The top bracket of the Instron machine was then used to actuate the plunger of the insertion device at the desired rate. The insertion device was then opened to again allow removal of the agarose well with embedded AB probe.

Electrode insertion tip spread and average depth were measured with a digital microscope (DMS1000, Leica Microsystems, Wetzlar, Germany), which provided a 1.0 mm scale bar on the display read out. Images were then saved, showing both a bottom and side view (see FIG. 8) of each inserted probe in the agarose gel, and processed to determine values for both maximum tip spread and average tip insertion depth. Electrode tip spread was defined as the minimum circle required to encompass all eight electrode tips, while insertion depth was defined by the average distance of each electrode tip from the surface of the agarose gel.

Results

Here the results of the insertion testing for each probe type (ten samples per probe type) are presented with respect to three metrics: maximum insertion force, electrode tip spread, and average electrode insertion depth. Metric comparison and determination of significance was accomplished through independent one-tailed t-tests.

Note that while the possibility of failure to penetrate the agarose gel was also watched out for, none of the thirty trials demonstrated this. This was as expected, as prior to physical experimentation, a finite element analysis buckling simulation was run on a single niobium microwire electrode in SolidWorks2017, which suggested that a load of more than two times the expected load during insertion would be required to cause buckling.

A. Insertion Force

To gain an understanding of the degree of acute insertional damage that each probe type is likely to cause, without conducting an in vivo study, insertion force was measured with a precision gram loadcell as each probe sample was driven into the agarose gel phantom at a rate of 600 μm/s to a depth of 4.5 mm. Literature suggests a strong link between acute insertional force and tissue damage, as well as a consequential link to increased FBR and probe encapsulation. Recording at 100 Hz was started approximately 10 seconds before each insertion to establish a baseline and continued for 5 seconds after. The maximum force recorded was then determined and averaged across each of the ten trials for each of the three probe types.

FIG. 8 presents sample photographs of (a) an inserted free probe, and (b) an inserted AB probe.

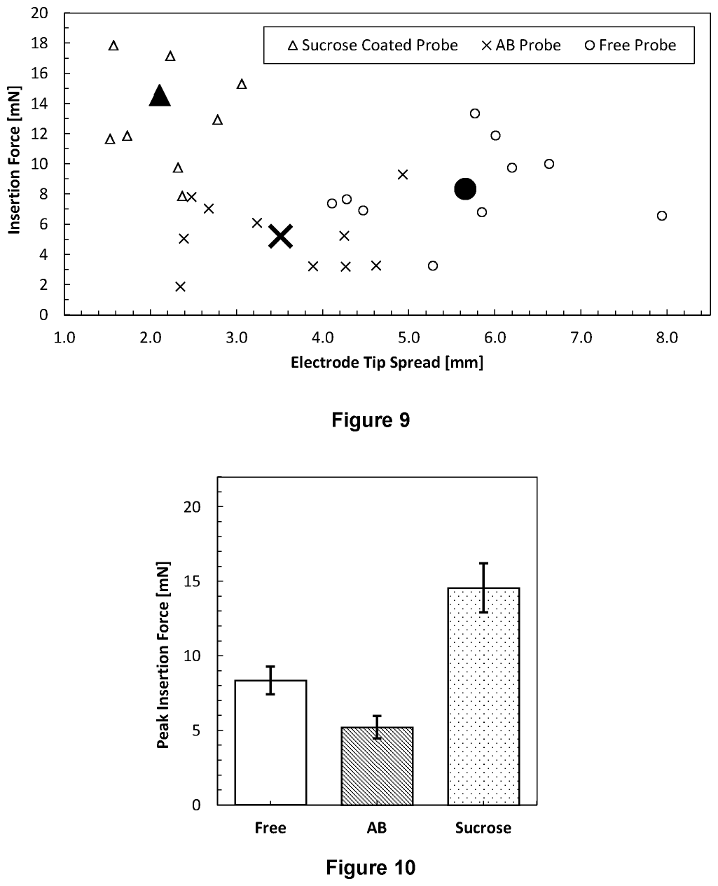

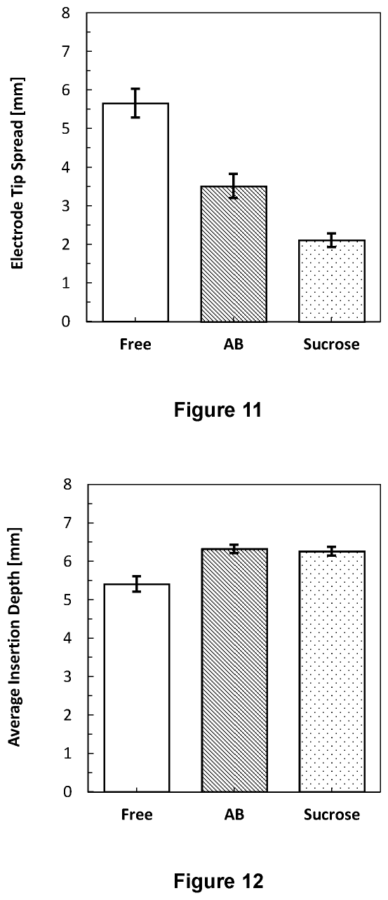

FIG. 9 is a scatter plot showing insertion force and electrode tip spread for the three different probe types (ten of each) into the agarose gel, with mean values for each probe type being shown by the enlarged respective symbol. FIG. 10 shows the mean peak insertion force for the three different probe types. The error bars in FIG. 10 (and likewise in FIGS. 11 and 12) represent standard error of the mean (SEM).

From FIGS. 9 and 10, the mean maximum insertion force for the device-inserted AB probes was shown to be significantly lower than that of both the free and sucrose-coated probes, (p<0.05) and (p<0.0005) respectively. Additionally, the mean maximum insertion force for the sucrose coated probe samples was shown to be higher than that of the free probes (p<0.005).

A Electrode Tip Spread

In the absence of electrode buckling, electrode spreading within the tissue during insertion was evaluated. Excessive spreading of electrodes, and a subsequent increase in relative electrode tip distances, can lead to decreased decoding accuracy. Thus, following all insertion tests for the three probe types (thirty total trials), the agarose gel wells were imaged under a digital microscope with the probes still inserted, Note that after removal from the Instron fixtures, each probe was manually inserted the remaining ˜2.5 mm until the probe head was flush with the surface of the agarose (or in contact with the AB plate in the case of the AB probes). Images were taken through the bottom window of the agarose gel wells and the electrode tips brought into focus. The smallest circle able to encompass all eight electrode tips was then drawn and the diameter recorded. As shown in FIG. 11, use of the AB probes and prototype insertion device had a significant effect on decreasing electrode tip spread when compared to free probe insertion (p<0.005). However, the sucrose-coated probes were able to achieve even less tip spread than the AB probes (p<0.005).

C. Electrode Insertion Depth

Finally, in conjunction with electrode tip spread, average electrode insertion depth for each probe was evaluated to characterize the degree of electrode bending during insertion. Reaching a reliable insertion depth is important for recording neurons in the desired layers of the cortex. (While in this study, all microwire electrodes were the same length, probes could also be constructed with multiple lengths of electrodes for multi-layer cortical recording.) Side view images were analysed of each probe sample after insertion into the agarose gel, and the average electrode tip depth calculated. As shown in FIG. 12, the AB probes, inserted with the prototype insertion device, were able to achieve a larger mean insertion depth when compared to free probe insertion (p<0.005), and a similar mean insertion depth to that of the sucrose-coated probes.

Conclusions of Tests and Summary of Findings

From FIGS. 9-12, the AB probe type was shown to have the lowest insertion force of the three types (p<0.05 vs free type and p<0.0005 vs sucrose-coated), while also achieving less electrode spread and deeper average insertion depth than the free probe type (p<0.005).

The present work has presented an insertion method for increasing the reliability of cortical insertion, while minimizing insertion force, for probes with multiple flexible microwire electrodes. Evidence in support of the method has been provided by an insertion study conducted on three types of mock probes, using 0.6% by weight agarose gel to simulate cortical tissue. The prototype device and probe architecture was shown to simultaneously decrease the amount of electrode tip spread and increase the average insertion depth, when compared to a probe with free and unsupported electrodes. While performing worse in these respects when compared to the competing sucrose-coated method, the presented method was able to maintain significantly lower insertion forces, which in a clinical setting is likely to result in less insertional damage and a subsequently less severe foreign body response.

Of note is that the AB probes achieved significantly lower insertion forces than the free probes as well. This is thought to be attributed to the lower insertion spread. Since the electrodes are inserted more linearly into the agarose gel, they cause less resistance during insertion than if they were spreading out on angled paths, as was the case with the free probes. The result is a lower insertion force and an expected lower incident of tissue damage. This is visually noticeable in FIG. 8, where considerably larger destruction of the agarose gel occurred near the base of the electrodes of the free probe (a), as opposed to the AB probe (b).

This combination of low insertion spread, high insertion depth, and low insertion force suggests that the presented device and probe architecture has great potential for clinical applications, with the potential for higher fidelity recording and decoding while also mitigating the FBR.

Example Manufacturing Techniques

The present AB plates 230 and AB probes 240 may be made using any of the following techniques (not an exhaustive list):
Laser cutting
Deep reactive-ion etching
3D printing (laser-sintering, stereolithography, fused deposition modelling, etc)
Micro-machining (micro-drilling, milling, turning, etc.)
Solvent Casting
Laser micro-machining (milling, drilling, etc.)
Injection moulding
Compression moulding
Low and High Temperature Cofired Ceramic Manufacturing (LTCC and HTCC)

The syringe-like insertion device 300 may be made using any of the following techniques (not an exhaustive list):
Injection Moulding
Compression Moulding
Machining (milling, turning, etc.)
3D printing (laser-sintering, stereolithography, fused deposition modelling, etc)
Investment/lost-wax casting
Extrusion moulding

The following materials may be used, as examples of biocompatible polymers, metals, and other suitable materials for machining/moulding/printing (again, not an exhaustive list):
Nylon
Polyetheretherketone (PEEK)
Delrin (acetyl)
Teflon
PEI (polyetherimide)
PPSUs (polyphenylsulfones) like Radel
PSUs (polysulfones) like Udel
Polycarbonate
Polypropylene (PP)
Polymethylmetacrylate (PMMA) (Acrylic)
Polyurethanes
Silicon (mostly just probe/AB plate)
Sapphire (just probe/AB plate)
Silicone
Tungsten
Platinum-iridium
Chromium alloys
Titanium alloys
Glass (syringe body)
Ceramics
Bioresorbable materials
Sucrose
Pyrolytic carbon
Pyrolytic graphite

It should be noted that, in some cases, the AB plates may be made from bioresorbable materials. Accordingly, such a plate may be left in place beneath the probe head once a probe has been inserted, and the plate will then be resorbed over time.

### Possible Modifications and Alternative Embodiments

Detailed embodiments have been described above, together with some possible modifications and alternatives. As those skilled in the art will appreciate, a number of additional modifications and alternatives can be made to the above embodiments whilst still benefiting from the inventions embodied therein.

The above embodiments primarily use a single AB plate 230 incorporating holes 232 for each microwire electrode 220 to pass through. Alternatively, as mentioned above, one could exploit a decrease in the unsupported length L by, instead of using one plate AB plate 230, using two slotted AB plates, wherein the parallel slots of one plate cross the parallel slots of the other plate, preferably orthogonally, such that each plate constrains a respective single axis of buckling. Such a slotted design allows for the removal of the AB plates prior to complete insertion, avoiding the need to make the electrodes longer than their required insertion depth.

FIGS. 13, 14 and 15 illustrate an insertion device 300a with a modified probe-loading tip 330a, for use with two crossing (preferably orthogonally-intersecting) slotted AB plates 230a, 230b. As shown in FIG. 13, the probe-loading tip 330a incorporates orthogonal channels for receiving the slotted plates 230a, 230b. Each of the slotted AB plates 230a, 230b is provided with a loop 231 or other handle means, by which the slotted plates 230a, 230b may be slid out of the probe-loading tip 330a.

Initially, both the slotted AB plates 230a, 230b are used to constrain the microwires 220. More particularly, the crossing slots of the two plates 230a, 230b cooperate to define vertical channels for constraining the microwires 220 at the positions where the slots cross. Preferably each microwire is constrained by a respective discrete vertical channel formed by the crossing slots of the two plates 230a, 230b. As the plunger 310 is depressed, the probe head 210 moves down, and the microwires 220 are inserted into the tissue, the uppermost slotted plate 230b can first be removed. Then, as the plunger 310 is further depressed, the probe head 210 moves further down, and the microwires 220 are inserted further into the tissue, the second slotted plate 230a can be removed. Ultimately, the microwires 220 can then be fully inserted into the tissue without any AB plate remaining in place—i.e. with the probe head 210 corning into contact with the tissue—thereby maximising the depth of penetration of the microwires 220 into the tissue, and minimising the thickness of entities on the surface of the tissue.

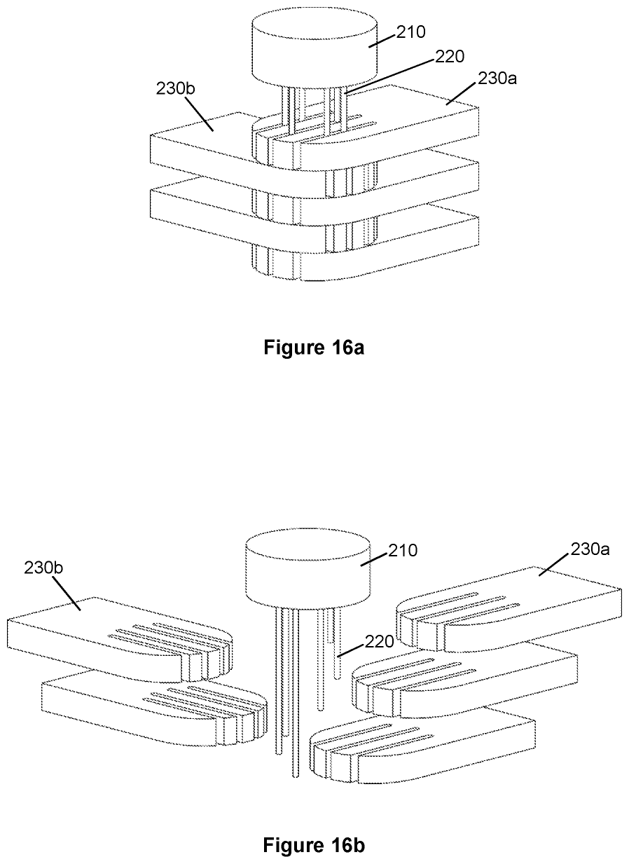

FIGS. 16a and 16b illustrate an alternative arrangement in which multiple slotted AB plates 230a, 230b are used to constrain different lengths of electrodes 220, the slotted AB plates 230a, 230b being sequentially removed during the insertion procedure, as the probe is inserted.

FIG. 17 illustrates another alternative arrangement in which a single AB plate 230c is used to provide selective support to a subset of longer electrodes 220b only. The AB plate 230c has a relief cut out to allow shorter (and less prone to buckling) electrodes 220a to bypass the AB plate 230c.

Thus, in practice, in the event that the electrodes are of different lengths, an assessment may be made as to which electrodes are of a length that may render them prone to buckling on insertion. Shorter electrodes 220a that do not require AB support may then be aligned with an appropriately-shaped cut-out region of the AB plate, such that they do not pass through apertures in the AB plate during the insertion process, whereas the longer electrodes 220b are arranged to pass through appropriately-positioned apertures in the AB plate.

FIG. 18 illustrates another alternative arrangement, in this case showing a multi-thickness probe head 210a and a counterpart multi-thickness AB plate 230d, which fit together in an complementary interlocking manner (like a puzzle) when fully inserted. The multiple steps of the probe head 210a and the AB plate 230d may be used to accommodate and constrain different lengths of electrodes 220. FIG. 19 shows the stepwise insertion procedure of the probe and AB plate of FIG. 18.

Informed by the present disclosure, other designs of probe heads and AB plates, based on the present principles, will be apparent to those skilled in the art.

Although the present insertion device 300 has been primarily described for use in inserting multi-microwire probes 200, it may alternatively be used for inserting other probes that require the controlled application of a linear downward force. It may be used to insert probes with various numbers, shapes, or materials of electrodes, as well as electrode-based electronic implants/devices for other purposes, that require the controlled application of linear downward force.

Thus, although the present probes have been primarily described as being multi-microwire probes 200, the present principles (i.e. using one or more AB plates to constrain one or more electrodes to prevent them from buckling, and the use of a complementary insertion device) are applicable to other designs of probes comprising one or more discrete slender electrodes that is/are prone to buckling. For instance, a probe having a single microwire electrode (or some other slender electrode) may be inserted using one or more of the present AB plates, using the present insertion device.

Such probes may be sensing probes (as may be used to detect brain activity, for example) or stimulating probes (as may be used to apply some kind of impulse to the brain, for example).

Finally, the although the present principles have been described in relation to the implantation of electrode-based probes for biological purposes, the present principles may be applied to the installation of other slender penetrating members into an underlying substrate, e.g. for medical/biological applications (e.g. the installation of catheters or cannulas) and applications in other areas of industry or research.

### CLOSING SUMMARY

Brain machine interfaces have the potential to improve the quality of life for millions of people suffering from neurological disorders and injuries around the world, yet are plagued with issues in achieving long term implanted recording stability. This is largely a result of increased electrode impedance and encapsulation over time. It has been shown that damage and inflammation caused during insertion by electrodes that are too large and stiff leads to a sustained inflammatory tissue response, commonly referred to as the foreign body response. Accordingly, neural interfaces with ever smaller and more flexible electrodes are continually in development, but unfortunately face problems of their own, first and foremost of which is buckling and bending during insertion. The present work presents an insertion method, an insertion device and a probe architecture, that promote straight insertion of a microwire probe without buckling, while simultaneously minimizing the insertion force for multi-microwire electrode probes. When compared against insertion of probes with unsupported free electrodes, the present method achieved significantly straighter electrode insertion, resulting in both a smaller distance between electrode recording tips and a greater average insertion depth. At the same time, the present method was able to maintain significantly lower insertion forces when compared to probes with sucrose coated electrodes, a common current technique for promoting reliable insertion without buckling. The present method has the potential to be adapted to any design or structure of neural interface and is expected to deliver long term recording stability.

1. A probe assembly comprising:
an electrode-based probe comprising a probe head and one or more slender electrodes extending from the probe head for insertion into biological tissue; and
a support element disposed around one or more of said electrodes, distal from the probe head, the support element comprising one or more apertures through which said one or more electrodes pass, the probe head and said electrode(s) being movable relative to the support element during insertion;
wherein the support element is configured to constrain the angle of the end of the said electrode(s) at the point of insertion into the tissue.

2. The probe assembly according to claim 1, wherein the support element is configured to constrain the end of the said electrode(s) so as to be orthogonal to the tissue at the point of insertion; and/or
wherein the support element is configured to constrain the end of the said electrode(s) so as to be linear with the rest of the electrode(s); and/or
wherein the support element is in the form of a plate; and/or
wherein the shape of the support element corresponds to the shape of the probe head; and/or
wherein the aperture(s) are in the form of one or more discrete holes, through each of which a respective electrode passes.

3-6. (canceled)

7. The probe assembly according to claim 1, wherein the underside of the probe head incorporates one or more protrusions or recesses for engaging with corresponding recesses or protrusions in the upper surface of the support element.

8. The probe assembly according to claim 1, wherein the support element is made of a bioresorbable material.

9. The probe assembly according to claim 1, wherein:
the support element is a first plate, and the aperture(s) are in the form of a slot or a plurality of parallel slots within the plate; and
the probe assembly further comprises a second such plate, also incorporating a slot or a plurality of parallel slots;
wherein the first and second plates are arranged one above the other, such that the slot(s) of the first plate cross the slot(s) of the second plate, the crossing points of the slots defining one or more channels for constraining said one or more electrodes during insertion.

10. The probe assembly according to claim 9, wherein the electrodes are of different lengths, and the probe assembly further comprises one or more additional slotted plates disposed around one or more relatively long electrodes.

11. The probe assembly according to claim 9, wherein each of the slotted plates comprises a handle for moving the respective plate in a direction parallel to the direction of the slot(s) within the plate, to remove the plate from the probe assembly.

12. The probe assembly according to claim 1, wherein the electrodes are a plurality of discrete microwire electrodes.

13.-18. (canceled)

19. An insertion device for inserting the electrode(s) of a probe assembly into biological tissue, the probe assembly comprising:
an electrode-based probe comprising a probe head and one or more slender electrodes extending from the probe head for insertion into biological tissue; and
a support element disposed around one or more of said electrodes, distal from the probe head, the support element comprising one or more apertures through which said one or more electrodes pass, the probe head and said electrode(s) being movable relative to the support element during insertion;
wherein the support element is configured to constrain the angle of the end of the said electrode(s) at the point of insertion into the tissue;
the insertion device comprising:
a holding part for holding the support element against the tissue or in close proximity to the tissue; and
an applicator for applying an insertion force to the probe head, to drive the probe head towards the support element and thereby cause the electrode(s) to move through the aperture(s) in the support element and become inserted into the tissue.

20. The insertion device according to claim 19, further comprising a device body having a probe-loading tip, the device body having a longitudinal channel therein, in communication with the probe-loading tip;
wherein the probe-loading tip is configured to receive and support the probe assembly;
wherein the holding part is provided by the probe-loading tip; and
wherein the applicator comprises a plunger located within the longitudinal channel, the plunger having a pushing part at one end, proximal to the probe-loading tip, the plunger being longitudinally advanceable within the channel so as to cause the pushing part to push the probe head in use.

21. The insertion device according to claim 20, wherein the probe-loading tip comprises a gripping member, such as an O-ring, for gripping the support element; and/or
wherein the probe-loading tip comprises a gripping member, such as an O-ring, for gripping the probe head.

22. (canceled)

23. The insertion device according to claim 20, wherein the plunger has a pushable head at the end of the plunger distal from the probe-loading tip, the length of the plunger being such that, when the plunger is fully depressed against the probe head and the support element, the distance by which the underside of the pushable head is proud of the top of the device body is greater than the combined thickness of the probe head and the support element.

24. The insertion device according to claim 23, wherein the device body further comprises a handle by which the device body can be raised towards the underside of the pushable head.

25. The insertion device according to claim 20, wherein the probe-loading tip comprises lateral slots through which slotted plates can be inserted to surround the electrode(s) and thereby form the probe assembly, and through which the slotted plates can be withdrawn during insertion of the electrode(s) into the tissue.

26. The insertion device according to claim 20, wherein the probe-loading tip is pre-loaded with the probe assembly.

27. The insertion device according to claim 20, wherein the probe-loading tip is detachable from, and reattachable to, the rest of the device body.

28. The insertion device according to claim 19, being for manual use.

29. The insertion device according to claim 19, being adapted for robotic actuation.

30. The probe assembly according to claim 1, wherein the probe assembly is pre-loaded into a probe-loading tip for use in an insertion device.

31. A method of inserting one or more electrodes of an electrode-based probe into biological tissue, using a probe assembly comprising:
an electrode-based probe comprising a probe head and one or more slender electrodes extending from the probe head for insertion into biological tissue; and
a support element disposed around one or more of said electrodes, distal from the probe head, the support element comprising one or more apertures through which said one or more electrodes pass, the probe head and said electrode(s) being movable relative to the support element during insertion;
wherein the support element is configured to constrain the angle of the end of the said electrode(s) at the point of insertion into the tissue.
